(19) United States
(12) Patent Application Publication
(10) Pub. No.: US 2016/0271047 A1
(43) Pub. Date: Sep. 22, 2016

(54) STYLING SPRAY HAVING A VOLUME EFFECT

(71) Applicant: Henkel AG & Co. KGaA, Duesseldorf (DE)

(72) Inventors: Thorsten Knappe, Schenefeld (DE); Pamela Kaftan, Hamburg (DE)

(73) Assignee: Henkel AG & Co. KGaA, Duesseldorf (DE)

(21) Appl. No.: 15/168,418

(22) Filed: May 31, 2016

Related U.S. Application Data
(63) Continuation of application No. PCT/EP2014/074917, filed on Nov. 18, 2014.

(30) Foreign Application Priority Data
Dec. 16, 2013 (DE) 10 2013 226 048.2

(51) Int. Cl. A61K 8/81; A61Q 5/06; A61K 8/34; A61K 8/04

(52) U.S. Cl. CPC A61K 8/8147; A61K 8/046; A61Q 5/06; A61K 8/345

(57) ABSTRACT

The invention relates to an aerosol composition, comprising a) a cosmetic styling product, including a1) at least one polyalcohol selected from the group comprising glycerol, propylene glycol, and panthenol, in particular glycerol, a2) polymer including carboxylic acid groups, wherein at least 50 wt % of the polymer including carboxylic acid groups is a copolymer formed by the monomers, a2i) N-tert.-octylacrylamide, a2ii) acrylic acid, a2iii) tert.-butylaminoethylmethacrylic acid and optionally further monomers, characterised in that the ratio of the parts by weight of component a1) and component a2) in the product is greater than 1, b) a propellant or mixture of propellants, when used for temporary deformation of keratin fibres are distinguished by improved sheen, good volume, and improved feel of the hair.

### FIELD OF THE INVENTION

The present invention generally relates to the specialist technical field of temporary reshaping of keratin-containing fibres, in particular human hair. The subject of the present invention is constituted by aerosol compositions including a cosmetic styling product having good properties and at least one propellant, and by the use of these compositions for the temporary deformation of keratin-containing fibres, and by a corresponding method of use.

### BACKGROUND OF THE INVENTION

Keratin-containing fibres are understood in principle to mean all animal hair, for example wool, horsehair, angora hair, furs, feathers, and products or textiles produced therefrom. However, the keratin fibres are preferably human hair.

A nice-looking hairstyle is nowadays generally regarded as an essential part of a well-groomed appearance. Time and time again, based on current fashion trends, hairstyles that can be constructed with many types of hair using only firming active ingredients or that stay in place for a relatively long period of time up to several days are considered chic. Hair treatment products that permanently or temporarily shape the hair therefore play an important role. Whereas, in the case of permanent reshaping, the chemical structure of the keratin-containing fibres is modified by reduction and oxidation, no such modification of the chemical structure takes place in the case of temporary reshaping. Temporary shaping is intended to provide good hold without having a detrimental effect on the healthy appearance of the hair, for example the sheen thereof. Corresponding products for the temporary deformation of keratin fibres, also referred to hereinafter as styling products, usually include synthetic polymers and/or waxes as fixing active ingredient.

Styling products can be packaged for example in the form of hairspray, hair wax, hair gel, hair mousse, etc. In particular, application in the form of a spray or a foam by means of an aerosol dispensing container has proven to be very popular, because the application from spray or pump containers is possible in a simple and clean manner.

In addition to a high degree of hold, however, styling products should satisfy a whole range of further requirements. Provision of shine, volume, and the improvement of the feel of the hair can be mentioned here in particular.

EP 1307175 B1 discloses a hairstyling product comprising a non-rigid emulsion-polymerised cross-linked silicone polymer and a further copolymer. This styling product is fractionated in the form of an aerosol hairstyling mousse or a hairstyling cream or gel and provides excellent style creation (high degree of hold) and also sensory feeling (good feel of the hair).

U.S. Pat. No. 8,574,553 B2 discloses a cosmetic hair composition having a water content of less than 10 wt %, which is packaged in an aerosol device. The composition comprises at least one anionic fixing polymer, at least one polyol, at least one liquid fatty alcohol, at least one C1-C4 monoalcohol, and one or more propellants. According to U.S. Pat. No. 8,574,553 B2, this composition can hold the hairstyle well and at the same time it can improve the volume and the sheen of the hair.

Although special styling products have in each case satisfied at least one of the above requirements, not a single one has yet satisfied all of these requirements to an optimal degree.

Moreover, the addition of a number of polymers is normally required in order to achieve the various requirements. However, if too many different polymers are used, this can result in a series of disadvantages. Thus, problems may arise in the formulation, for example because the polymers react with one another or with other constituents of the product and precipitation or decomposition occurs. Certain polymers also tend to deposit so persistently on the skin and in particular on the hair that they can no longer be fully removed and lead to an undesirable accumulation of the polymer and therefore ultimately load the skin or hair.

The development of styling products that have all desired properties/requirements, specifically the high degree of hold, the shine, the volume and the improvement of the feel of the hair in combination, without forgoing compatibility and ease of removal, still presents difficulties, particularly when the application is to be fractionated into a light and clean form, such as an aerosol form. The reason for this is that the aerosol product needs to be capable of being easily distributed to the keratin-containing fibres, i.e. in the case of products in the form of an aerosol spray, the composition should be capable of being applied evenly as a fine spray mist in a targeted manner.

The polymers used in styling products of this type should therefore be soluble in non-aqueous or anhydrous organic solvents, but the films obtained from styling products of this type should usually be either water-soluble or dispersible in water in order to facilitate the ease of removal of said films from the hair. In addition, polymers of this type should be fully compatible with the solvents and/or propellants usually used in this styling product. Furthermore, the used polymers should have little or no tendency to react with the aromatic substances or other constituents possibly present in styling products.

The object of the present invention was therefore to provide a product that is stable in storage for the temporary deformation of keratin-containing fibres, which product is characterised by a high degree of hold and at the same time improves the sheen of the hairstyle, volume, and the feel of the hair.

The product can be applied to the fibres in particular as a fine, targeted spray mist and it should be easily removable by water and/or soap or shampoo. When stored in a metal aerosol container, only a small, and at best no corrosion tendency should be caused by the packaging of the product.

Furthermore, other desirable features and characteristics of the present invention will become apparent from the subsequent detailed description of the invention and the appended claims, taken in conjunction with this background of the invention.

### BRIEF SUMMARY OF THE INVENTION

An aerosol composition comprising a cosmetic styling product containing at least one polyalcohol selected from the group consisting of glycerol, propylene glycol and panthenol, in particular glycerol, and polymer including carboxylic acid groups, wherein at least 50 wt % of the polymer including carboxylic acid groups is a copolymer formed by the monomers a2i) N-tert.-octylacrylamide, a2ii) acrylic acid, a2iii) tert.-butylaminoethylmethacrylic acid and optionally further monomers, characterised in that the ratio of the parts by weight of component a1) and component a2) in the product is greater than 1; and a propellant or mixture of propellants.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the present invention is an aerosol composition comprising
a) a cosmetic styling product including
   a1) at least one polyalcohol selected from the group consisting of glycerol, propylene glycol and panthenol, in particular glycerol,
   a2) polymer including carboxylic acid groups, wherein at least 50 wt % of the polymer including carboxylic acid groups is a copolymer formed by the monomers
      a2i) N-tert.-octylacrylamide,
      a2ii) acrylic acid,
      a2iii) tert.-butylaminoethylmethacrylic acid
      and optionally further monomers,
   characterised in that the ratio of the parts by weight of component a1) and component a2) in the product is greater than 1,
b) a propellant or mixture of propellants.

A key constituent of the aerosol compositions according to the invention is the polymer included in the cosmetic styling product, which polymer comprises at least one carboxylic acid group and in the context of the invention within the scope of the temporary reshaping of keratin-containing fibres contributes to the hold of the shaped form of the fibres (in the case of hair in particular the hold of a hairstyle or the hair volume). What is known as the curl-retention test is often used as a test method for the fixing effect of an active ingredient.

Preferred polymers a2) are based on at least one monomer from the group of acrylic acid, methacrylic acid, C₁-C₆ alkyl acrylic esters, C₁-C₆ alkyl methacrylic esters. The acrylic esters and methacrylic esters are preferably esters of the respective acids with non-tertiary alkyl alcohols having alkyl groups from 1 to 12 carbon atoms, especially 2 to 4 carbon atoms. For example, ethyl acrylate, ethyl methacrylate, propyl acrylate, propyl methacrylate, n-butyl acrylate, n-butyl methacrylate, isobutyl acrylate, 2-methylbutyl acrylate, 2-ethylhexyl acrylate, n-octyl acrylate, isooctyl acrylate, isooctyl methacrylate, isononyl acrylate and isodecyl acrylate may be mentioned as suitable monomers.

The group constituted by these film-forming and/or fixing polymers a2) includes, inter alia, a copolymer formed from the monomers N-tert.-octylacrylamide, acrylic acid, tert.-butylaminoethylmethacrylic acid and optionally other monomers, which copolymer can be present in an amount of at least 50 wt % in the polymer a2). The copolymer described above is sold for example under the name Amphomer® (INCI name: octylacrylamide/acrylates/butylaminoethyl methacrylate copolymer) by the company National Starch.

An aerosol composition that is preferred in accordance with the invention is characterised in that at least 60 wt %, preferably at least 70 wt %, particularly preferably at least 90 wt %, and in particular at least 95 wt %, and very particularly 100 wt % of the polymer a2) is a copolymer formed by the monomers
a2i) N-tert.-octylacrylamide,
a2ii) acrylic acid,
a2iii) tert.-butylaminoethylmethacrylic acid
and optionally further monomers.

A further very particularly preferred aerosol composition according to the invention includes, as component a2), at least one polymer known under the INCI name: octylacrylamide/acrylates/butylaminoethyl methacrylate copolymer. A further very particularly preferred aerosol composition according to the invention includes, as component a2), exclusively a polymer known under the INCI name: octylacrylamide/acrylates/butylaminoethyl methacrylate copolymer. The use of this polymer has proven to be advantageous for the cosmetic properties and the stability in storage of the products according to the invention.

In the context of the invention the specified weight quantities relating to the “styling product” are calculated on the basis of the total weight of the styling product (i.e. without the propellant/mixture of propellants).

An aerosol composition that is preferred in accordance with the invention is characterised in that the part by weight of component a2) in the total weight of the cosmetic styling product is between 0.1 and 20 wt %, preferably between 0.5 and 15.0 wt %, and in particular between 1.0 and 10.0 wt %.

A further key constituent of the aerosol compositions according to the invention is the polyalcohol included in the cosmetic styling product, said polyalcohol being selected from the group consisting of glycerol, propylene glycol, and panthenol, in particular glycerol. Such polyalcohols a1) are plasticisers of the polymers a2) and increase the flexibility of the polymer film formed by applying the composition according to the invention.

A very particularly preferred aerosol composition according to the invention includes glycerol as component a1) in the cosmetic styling product.

The aerosol composition according to the invention is characterised in that the part by weight of component a1) in the total weight of the cosmetic styling product is between 0.5 and 30 wt %, preferably between 1.0 and 25 wt %, and in particular between 2.0 and 15 wt %.

A ratio of the parts by weight of component a1) and component a2) in the cosmetic styling product between 1:1 and 20:1, preferably between 2:1 and 10:1, has proven to be particularly advantageous for the cosmetic properties of the aerosol composition according to the invention. Aerosol compositions in which the ratio of the parts by weight of component a1) and a2) in the cosmetic styling product is between 1:1 and 20:1, preferably between 2:1 and 10:1, are therefore preferred in accordance with the invention.

A further very particularly preferred aerosol composition according to the invention includes glycerol as component a1) and octylacrylamide/acrylates/butylaminoethyl methacrylate copolymer as component a2) in the cosmetic styling product, wherein the ratio of the parts by weight of component a1) and component a2) in the product is greater than 1.

It should be noted that the polymers used in accordance with the invention can be partially neutralised before they are incorporated into the finished styling product, thereby allowing them to be removed from the hair by simply rinsing with water. This can be done in such a way that the polymers are reacted in the form of a solution in an organic solvent with or without addition of water with an alkaline reagent at a concentration equivalent on a molar basis to a minimum of approximately 50% of the available carboxyl groups present in the polymers. Suitable alkaline materials that can be used for this purpose are for example sodium hydroxide and potassium hydroxide, ammonia, primary, secondary and tertiary amines, alkanolamines, and hydroxyamines such as amonimethyl propanol and aminomethyl propandeiol. If the polymers are not pre-neutralised in this way, their potential removal can then still be carried out in a simple manner by using a weakly alkaline solution, for example using a soap solution.

An aerosol composition that is preferred in accordance with the invention includes in the cosmetic styling product, based on the total weight thereof, a total from 0.05 to 10 wt %, preferably 0.1 to 8 wt %, more preferably 0.2 to 5 wt %, and in particular 0.3 to 2 wt % of alkaline materials, preferably aminomethyl propanol.

Besides the cosmetic styling product a), the aerosol composition according to the invention comprises a propellant (mixture) b) as further constituent.

The propellants that are preferred in accordance with the invention are selected from the hydrocarbons having 3 to 5 carbon atoms, such as propane, n-butane, iso-butane, n-pentane and iso-pentane, dimethyl ether, carbon dioxide, nitrous oxide, fluorocarbons and chlorofluorocarbons, and mixtures of these substances. Very particularly preferred propellants are propane, butane, isobutane, pentane, isopentane, dimethyl ether and mixtures of these above-mentioned propellants in each case with each other.

In accordance with a preferred embodiment, the aerosol composition according to the invention includes the above-mentioned hydrocarbons, dimethyl ether, or mixtures of said hydrocarbons with dimethyl ether, as the sole propellant. However, the invention also explicitly includes the co-use of propellants of the chlorofluorocarbon type, but particularly fluorocarbons.

An aerosol composition that is preferred in accordance with the invention is characterised in that the propellant or mixture of propellants is selected from dimethyl ether, alkanes having 3 to 5 carbon atoms, and fluorocarbons.

A particularly preferred aerosol composition is characterised in that the propellant or mixture of propellants is used in amounts from 10 to 85 wt %, preferably 20 to 80 wt %, and in particular 35 to 60 wt %, based on the total aerosol composition.

The aerosol composition according to the invention is preferably packaged in a pressurised container. A “pressurised container” is, in accordance with the invention, a container which inside has a higher gas pressure than outside the container and from which a gas flow can be removed via a valve. A pressurised container with the aid of which a product (for example a liquid composition) is discharged via a valve by means of the internal gas pressure of the container is known according to definition as an “aerosol dispensing container” or “aerosol container”.

The aerosol compositions according to the invention can be produced in the usual way. Generally, all constituents of the preparation of the aerosol composition according to the invention are introduced into a suitable pressure-resistant container. This container is then sealed using a suitable spray head/valve. Lastly, the desired amount of the specific propellant is sealed via conventional techniques.

Vessels made of metal (aluminium, tinplate, tin), protected or non-splintering plastic, or made of glass coated on the outside with plastic are conceivable pressure-resistant containers, and the selection of such a vessel is influenced by pressure resistance and breaking strength, resistance to corrosion, ease of filling, and also aesthetic considerations, ease of handling, suitability for printing, etc. Special internal protective coatings ensure the corrosion resistance with respect to the product packaged in the pressurised container. The used valves particularly preferably have an internally coated valve poppet, wherein the coating and valve material are compatible with one another. If aluminium valves are used, the valve puppets thereof can thus be coated internally for example using the coating Micoflex. If tinplate valves are used in accordance with the invention, the valve poppets thereof can thus be coated internally for example by PET (polyethylene terephthalate).

The compositions according to the invention can also be packaged in a multi-chamber dispenser. The multi-chamber dispenser can also be used such that one chamber is filled by the compressed propellant and another chamber is filled by the rest of the constituents of the aerosol composition according to the invention. A multi-chamber dispenser of this type is what is known as a bag-in-can packaging, for example.

The aerosol containers preferably have a stem valve having a stem bore from 1×0.2 mm to 1×0.7 mm.

The valves in turn preferably have a VPH bore from 0.00 mm to 0.60 mm.

The valves in turn preferably have an RTP diameter from 0.30 to 1.60 mm.

Here, the following valves are preferably suitable in accordance with the invention:
Coster type KPM (stem bore from 1×0.27 mm to 1×0.60 mm)
Coster type KRA (stem bore from 1×0.27 mm to 1×0.60 mm, in combination with VPH bores from 0.00 mm to 0.60 mm and RTP diameters from 0.60 to 1, 60 mm)
Coster type RKRA (stem bore from 1×0.27 mm to 1×0.60 mm, in combination with VPH bores from 0.00 mm to 0.60 mm and RTP diameters from 0.30 to 1.60 mm)
Coster type T (stem bore from 1×0.30 mm to 1×0.70 mm)
Coster type TRA (stem bores from 1×0.30 mm to 1×0.70 mm, in combination with VPH bores from 0.00 mm to 0.60 mm and RTP diameters from 0.30 to 1.60 mm)
Coster type RTRA (stem bores from 1×0.30 mm to 1×0.70 mm, in combination with VPH bores from 0.00 mm to 0.60 mm and RTP diameters from 0.30 to 1.60 mm)
Coster type KEN (stem bore from 1×0.27 to 1×0.60 mm)
Coster type RKEN (stem bore from 1×0.27 mm to 1×0.60 mm, in combination with VPH bores from 0.00 mm to 0.60 mm and RTP diameters from 0.30 to 1.60 mm)
Precision Standard (with stem bores 0.010″ to 0.024″ in combination with base openings from 0.018″ to 0.040″)
Precision tilting valve (with stem bores 0.010″ to 0.024″ in combination with base openings from 0.018″ to 0.040″)
SeaquistPerfect Ariane VX or Ariane XT (stem bore from 1×0.25 mm to 1×0.60 mm, in combination with VPH bores from 0.33 mm to 0.80 mm, and housing bores from 0.40 to 1.60 mm).

Spray heads having swirl nozzles, or particularly preferably what are known as mechanical break up (MBU) swirl nozzles, such as the nozzle V06.212 from the company Coster, the MBU soft swirl nozzle 0.020″ from the company Precision, or the spray head WAX with DU 25 and 27, are preferably suitable in accordance with the invention for providing an aerosol spray according to the invention.

Depending on the spray valve, spray rates in the embodiment as aerosol spray, based on a fully filled container, from 1.0 to 20.0 g/10 s are possible, but preferably from 6.5 to 10.0 g/10 s.

Here, the spray rate is determined such that an aerosol container filled with propellant gas and the corresponding styling product and sealed by the relevant valve is first weighed at room temperature (approximately 23° C.). The container, inclusive of its contents, is vigorously shaken ten times by hand, so that the contents mix thoroughly. The valve of the upright container is then actuated for 10 s. The container is then weighed again. The process is carried out five times in succession, and the statistical average from the results is formed. The difference between the two weighings is the spray rate per 10 s. From this, the spray rate per second can also be determined by simple division.

A very particularly preferred aerosol composition according to the invention is characterised in that it is present packaged in an aerosol container having a spraying device.

The aerosol composition is produced in accordance with the invention by mixing the film-forming polymers with an organic solvent different from the propellant (mixture). Such solvents usually comprise a mixture of alcohols. In some cases, a system consisting just of one alcohol can also be used. Water usually is not provided, but can be present in some preparations, preferably in small amounts.

In particular, the lower alcohols having 1 to 4 carbon atoms usually used for cosmetic purposes, for example ethanol and isopropanol, can in particular be included as alcohols. Organic solvents or a mixture of solvents can be included as additional co-solvents. Particularly suitable additional co-solvents are benzyl alcohol, phenoxyethanol, unbranched or branched hydrocarbons such as pentane, hexane, isopentane, and cyclic hydrocarbons such as cyclopentane and cyclohexane. In addition to their solubility properties, the main advantages of these solvents are their capability to dry quickly and their minimal effect on the aerosol container.

An aerosol composition that is preferred in accordance with the invention is characterised in that the cosmetic styling product, based on its total weight, includes 20 to 95 wt %, preferably from 40 to 90 wt %, and in particular from 65 to 85 wt % of an organic solvent different from the propellant (mixture), in particular ethanol or isopropanol, or a mixture thereof.

A further aerosol composition that is preferred in accordance with the invention is characterised in that the cosmetic styling product, based on its total weight, has a water content from 0.5 to 20 wt %, preferably from 1.0 to 15 wt %, and in particular from 1.0 to 10 wt %.

The styling product according to the invention preferably additionally includes at least one surface-active substance, wherein both anionic and zwitterionic, ampholytic, non-ionic and cationic surface-active substances are suitable in principle. The surface-active substances comprise substantially two groups, i.e. surfactants and emulsifiers, which are treated here jointly.

An aerosol composition that is preferred in accordance with the invention is characterised in that the cosmetic styling product, based on its total weight, includes 0.01 to 5.0 wt %, preferably 0.02 to 4.0 wt %, and in particular 0.05 to 2.0 wt % of surfactants and/or emulsifiers.

All anionic surface-active substances suitable for use on the human body of suitable as an ionic surfactants and emulsifiers in products according to the invention. These substances are characterised by a water-solubilizing anionic group, such as a carboxylate, sulfate, sulfonate or phosphate group and a lipophilic alkyl group having approximately 8 to 30 C atoms. Glycol groups or polyglycol ether groups, ester groups, ether groups and amide groups and also hydroxyl groups can additionally be included in the molecule. Examples of suitable anionic surfactants and emulsifiers are
linear and branched fatty acids having 8 to 30 carbon atoms (soaps),
ether carboxylic acids of formula (T-I)

R¹O—(CH₂CH₂O)ₓ—CH₂—COOH (T-I)

in which R¹ is a linear alkyl group having 8 to 30 carbon atoms and x=0 or 1 to 16.
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic acid mono- and dialkyl esters having 8 to 24 carbon atoms in the alkyl group and sulfosuccinic acid monoalkylpolyoxyethyl esters having up to 8 carbon atoms in the alkyl group and 1 to 6 oxyethyl groups, preferably 1 to 4 oxyethyl groups,
linear alkane sulfonates having 8 to 24 carbon atoms,
linear alpha-olefin sulfonates having 8 to 24 carbon atoms,
alpha-sulfo fatty acid methyl esters of fatty acids having 8 to 30 carbon atoms,
alkyl sulfates and alkyl polyglycol ether sulfates of the formula (T-II)

R²—O(CH₂—CH₂O)ₓ—SO₃H (T-II)
in which R² is a preferably linear alkyl group having 8 to 30 carbon atoms and x=0 or 1 to 14,
mixtures of surface-active hydroxy sulfonates,
sulfated hydroxyalkylpolyethylene glycol ethers and/or hydroxyalkylene propylene glycol ethers,
sulfonates of unsaturated fatty acids having 8 to 24 carbon atoms and 1 to 6 double bonds,
esters of tartaric acid and citric acid with alcohols which represent addition products of approximately 2 to 15 molecules of ethylene oxide and/or propylene oxide onto fatty alcohols having 8 to 22 carbon atoms,
alkyl ether phosphates and/or alkenyl ether phosphates of the formula (T-III),

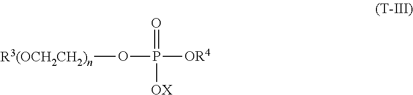

in which R³ is preferably an aliphatic hydrocarbon group having 8 to 30 carbon atoms, X is hydrogen, an alkali metal or alkaline earth metal or NR⁵R⁶R⁷R⁸, with R⁵ to R⁸ denoting, independently of one another, a C₁ to C₄ hydrocarbon group, R⁴ is hydrogen, a group R³(CH₂CH₂O)ₙ or X, and n=1 to 10,
sulfated fatty acid alkylene glycol ester of the formula (T-IV)

R⁹CO-(Alk-O)n—SO₃M (T-IV)
in which R⁹ denotes a linear or branched, aliphatic, saturated and/or unsaturated acyl group having 6 to 22 carbon atoms, Alk denotes CH₂CH₂, CHCH₃CH₂ and/or CH₂CHCH₃, n denotes numbers from 0.5 to 5, and M denotes a cation, as are described in DE-OS 197 36 906.5,
monoglyceride sulfates and monoglyceride (ether) sulfates of formula (T-V),

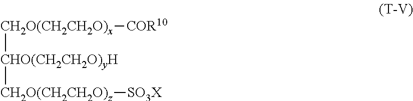

in which R¹⁰ denotes a linear or branched acyl group having 6 to 22 carbon atoms, x, y and z together denote 0 or numbers from 1 to 30, preferably 2 to 10, and X denotes an alkali metal or alkaline earth metal. Typical examples of monoglyceride (ether) sulfates suitable in the context of the invention in the form of their sodium salts are the reaction products of lauric acid monoglyceride, coconut fatty acid monoglyceride, palmitic acid monoglyceride, stearic acid monoglyceride, oleic acid monoglyceride and tallow fatty acid monoglyceride, and ethylene oxide adducts thereof with sulfur trioxide or chlorosulfonic acid.

Monoglyceride sulfates of formula (T-V) are preferably used, in which R¹⁰ denotes a linear acyl group having 8 to 18 carbon atoms.

Zwitterionic surfactants and emulsifiers are considered to be constituted by surface-active compounds that carry, in the molecule, at least one quaternary ammonium group and at least one —COO⁽⁻⁾ or —SO₃⁽⁻⁾ group. Particularly suitable zwitterionic surfactants and emulsifiers are what are known as the betaines, such as N-alkyl-N,N-dimethylammonium glycinates, for example coconut alkyl dimethyl ammonium glycinate, N-acyl-aminopropyl-N,N-dimethyl ammonium glycinate, cocoacylaminopropyl dimethyl ammonium glycinate, and 2-alkyl-3-carboxymethyl-3-hydroxyethyl imidazolines containing in each case 8 to 18 carbon atoms in the alkyl or acyl group as well as coco acyl amino ethyl hydroxyethyl carboxymethyl glycinate. Preferred zwitterionic surfactants are the carboxylic acid amide derivative known under the INCI name cocamidopropyl betaine, and alkyl betaines having 10 to 20 carbon atoms in the alkyl group.

Ampholytic surfactants and emulsifiers are understood to be surface-active compounds which, in addition to a C₈-C₂₄ alkyl or acyl group, include at least one free amino group and at least one —COOH or —SO₃H group and are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyltaurines, N-alkylsarcosines, 2-alkylaminopropionic acids, and alkylamino acetic acids with in each case about 8 to 24 C atoms in the alkyl group. Particularly preferred ampholytic surfactants are N-cocoalkylaminopropionate, cocoacylaminoethylaminopropionate and C₁₂-C₁₈ sarcosine.

In accordance with the invention, all conventional cationic surfactants known to a person skilled in the art can be used. These are, in particular, quaternary ammonium compounds, esterquats, amines and/or cationised amines, amidoamines and/or cationised amidoamines.

Preferred quaternary ammonium compounds are ammonium halides, in particular chlorides and bromides, such as alkyltrimethylammonium chlorides, dialkyldimethylammonium chlorides and trialkylmethylammonium chlorides, for example cetyltrimethylammonium chloride, stearyl trimethyl ammonium chloride, distearyl dimethyl ammonium chloride, lauryl dimethyl ammonium chloride, lauryl dimethyl benzyl ammonium chloride and tricetylmethylammonium chloride, as well as the imidazolium compounds known under the INCI names quaternium 27, quaternium 83 and quaternium 87. The alkyl chains of the aforementioned surfactants preferably have 10 to 18 carbon atoms.

Esterquats are known substances which include both at least one ester function and at least one quaternary ammonium group as structural element. Preferred esterquats are quaternised ester salts of fatty acids with triethanolamine, quaternised ester salts of fatty acids with diethanol alkyl amines, and quaternised ester salts of fatty acids with 1,2-dihydroxypropyl dialkylamines. Such products are sold for example under the trade names Stepantex®, Dehyquart®, and Armocare® and Quartamin®.

The alkylamidoamines are usually prepared by amidation of natural or synthetic fatty acids and fatty acid cuts with dialkylaminoamines. A particularly suitable compound from this group of substances is the stearamidopropyldimethylamine available commercially under the name Tegoamid® S 18.

The anion of all previously described cationic compounds is selected from the physiologically acceptable anions. Examples of this are the halide ions, fluoride, chloride, bromide, sulfate of general formula RSO₃—, in which R means saturated or unsaturated alkyl groups having 1 to 4 carbon atoms, or anionic groups of organic acids, such as maleate, fumarate, oxalate, tartrate, citrate, lactate or acetate.

The non-ionic surfactants have, as hydrophilic group, for example a polyol group, a polyalkylene glycol ether group, or a combination of polyol and polyglycol ether groups. Non-ionic surfactants and non-ionic emulsifiers that are preferred in accordance with the invention are, for example,
addition products of 2 to 50 mol ethylene oxide and/or 0 to 5 mol propylene oxide onto linear and branched fatty alcohols having 8 to 30 carbon atoms, onto fatty acids having 8 to 30 C atoms and onto alkylphenols having 8 to 15 carbon atoms in the alkyl group,
addition products, capped at the end group with a methyl or C₂ to C₆ alkyl group, of 2 to 50 mol ethylene oxide and/or 0 to 5 mol propylene oxide onto linear and branched fatty alcohols having 8 to 30 carbon atoms onto fatty acids having 8 to 30 carbon atoms, and onto alkylphenols having 8 to 15 carbon atoms in the alkyl group, such as the types obtainable under the trade names Dehydol® LS, Dehydol® LT (Cognis),
C₁₂-C₃₀ carboxylic acid mono- and diesters of addition products of 1 to 30 mol ethylene oxide onto glycerol,
addition products of 5 to 60 mol ethylene oxide onto castor oil and hydrogenated castor oil,
polyol fatty acid esters, such as the commercial product Hydagen® HSP (Cognis) or Sovermol types (Cognis)
alkoxylated triglycerides,
alkoxylated fatty acid alkyl esters of the formula (T-VI)

R¹¹CO—(OCH₂CHR¹²)_{w}OR¹³ (T-VI)
in which R¹¹ denotes a linear or branched, saturated and/or unsaturated acyl group having 6 to 22 carbon atoms, R¹² denotes hydrogen or methyl, R¹³ denotes linear or branched alkyl groups having 1 to 4 carbon atoms, and w denotes numbers from 1 to 20,
amine oxides,
hydroxy mixed ethers, as described for example in DE-OS 19738866,
sorbitan fatty acid esters and addition products of ethylene oxide onto sorbitan fatty acid esters, such as polysorbates,
sugar fatty acid esters and addition products of ethylene oxide onto sugar fatty acid esters,
fatty acid-N-alkyl glucamides,
pegylated oils,
glycoside-substituted silicones.

Non-ionic surface-active substances are preferably selected as surfactants and/or emulsifiers.

Water-in-oil emulsifiers, in particular a very wide variety of pegylated oils, such as PEG-30 hydrogenated castor oil, PEG-35 hydrogenated castor oil, PEG-40 hydrogenated castor oil and PEG-60 hydrogenated castor oil, as well as water-in-oil emulsifiers based on silicone are particularly preferred in accordance with the invention.

A group of water-in-oil emulsifiers based on silicone that is particularly preferred in accordance with the invention is constituted by poly-(C2-C3)alkylene glycol-modified silicones, whose earlier INCI name dimethicone copolyol was, with the latest INCI names, PEG-x dimethicone (with x=2-20, preferably 3-17, particularly preferably 11-12), bis-PEG-y dimethicone (with y=3-25, preferably 4-20), PEG/PPG a/b dimethicone (where a and b are, independently, numbers from 2 to 30, preferably 3 to 30 and particularly preferably 12 to 24, in particular 14 to 20), bis-PEG/PPG-c/d dimethicone (where c and d are, independently, a number from 10 to 25, preferably 14 to 20, and particularly preferably 14 to 16), and bis-PEG/PPG-e/f PEG/PPG g/h dimethicone (where e, f, g and h, independently of one another, denote numbers from 10 to 20, preferably 14 to 18 and particularly preferably 16).

Particularly preferred water-in-oil emulsifiers according to the invention are selected from the group of alkoxylated silicone emulsifiers, preferably from the group of ethoxylated and propoxylated silicone emulsifiers. A substance from the group of PEG/PPG-n/m dimethicone with n+m>11, in particular with n+m between 16 and 46, preferably with n+m between 26 and 46, is particularly preferably used as water-in-oil emulsifier. The PEG/PPG-18/18 dimethicone commercially available for example in a 1:9 mixture with cylomethicone under the name Dow Corning 3225 C or Dow Corning 5225 C, the PEG/PPG-18/18 dimethicone commercially available for example in a 1:3 mixture with non-volatile dimethicone as Dow Corning ES 5227 DM Formulation Aid, the PEG/PPG-4/12 dimethicone available for example under the name Abil B 8852, and the bis-PEG/PPG-14/14 dimethicone commercially available for example in a mixture with cyclomethicone as Abil EM 97 (Evonik), the bis-PEG/PPG-20/20 dimethicone available for example under the name Abil B 8832, and the PEG/PPG-20/23 dimethicone (for example Silsoft 430 and Silsoft 440) are very particularly preferred.

Further W/O emulsifiers based on silicone that are preferred in accordance with the invention are poly(C2-C3)alkylene glycol-modified silicones that are hydrophobically modified with C4-C18 alkyl groups, particularly preferably cetyl PEG/PPG-10/1 dimethicone (formerly: cetyl dimethicone copolyol, available for example as Abil EM 90 or in a mixture of polyglyceryl-4 isostearate, cetyl PEG/PPG-10/1 dimethicone and hexyl laurate sold under the trade name Abil WE 09), and also alkyl methicone copolyol.

When selecting non-ionic oil-in-water emulsifiers that are suitable in accordance with the invention, it is particularly preferable to use a mixture of non-ionic oil-in-water emulsifiers in order to optimally adjust the stability of styling products according to the invention.

A preferred subject matter of the present invention is an aerosol composition comprising
a) a cosmetic styling product including
   a1) at least one polyalcohol selected from the group consisting of glycerol, propylene glycol and panthenol, in particular glycerol,
   a2) polymer including carboxylic acid groups, wherein at least 50 wt % of the polymer including carboxylic acid groups is a copolymer formed by the monomers
      a2i) N-tert.-octylacrylamide,
      a2ii) acrylic acid,
      a2iii) tert.-butylaminoethylmethacrylic acid
      and optionally further monomers,
   a3) at least one water-in-oil emulsifier,
   characterised in that the ratio of the parts by weight of component a1) and component a2) in the product is greater than 1,
b) a propellant or mixture of propellants.

Additives can also be incorporated as appropriate into the aerosol composition according to the invention in order to modify certain properties. Examples of these additives are UV light-protection filters with effect in the range of UV-A, UV-B and UV-C light as oil-soluble filters, dyes, perfume oils, fragrances and scents.

To summarise, the aerosol compositions according to the invention have a particularly high resistance to corrosion in an aerosol container, which is a great advantage compared with the prior art. The styling products used in accordance with the invention also have excellent stability in storage and excellent cosmetic properties. It is as advantageous in particular that the sheen of the hairstyle, the volume and the feel of the hair are also improved by the use of the aerosol composition according to the invention for the temporary deformation of keratin fibres.

A further subject of the present invention is the use of an aerosol composition according to the invention for the temporary deformation of keratin fibres, wherein the sheen effect, volume and feel of the hair are improved.

With regard to preferred embodiments of the uses according to the invention, that which has been stated in relation to the products according to the invention applies mutatis mutandis.

A further subject of the present invention is a method for treating hair, in which an aerosol composition according to the invention is applied to the hair and is left there until the hair is next washed.

With regard to preferred embodiments of the methods according to the invention, that which has been stated in relation to the products according to the invention applies mutatis mutandis.

The following table includes examples of (propellant-free) styling products according to the invention. All numerical values correspond, unless otherwise specified, to the amounts of the active ingredient in question in weight percent based on the total weight of the styling product:

| | Formula 1 | Formula 2 | Formula 3 | Formula 4 | Formula 5 |
|---|---|---|---|---|---|
| Polyalcohol a1)¹⁾ | 0.5 to 30 | 1.0 to 25 | 1.5 to 20 | 2.0 to 15 | 3.0 to 10 |
| Polymer a2)²⁾ | 0.1 to 20 | 0.5 to 15 | 1.0 to 12 | 1.0 to 10 | 2.0 to 7.0 |
| Further ingredients | ad 100 | ad 100 | ad 100 | ad 100 | ad 100 |
| | | | | | Formula |
| | Formula 6 | Formula 7 | Formula 8 | Formula 9 | 10 |
| Glycerol | 0.5 to 30 | 1.0 to 25 | 1.5 to 20 | 2.0 to 15 | 3.0 to 10 |
| octylacrylamide/ | 0.1 to 20 | 0.5 to 15 | 1.0 to 12 | 1.0 to 10 | 2.0 to 7.0 |
| acrylates/ | | | | | |
| butylaminoethyl | | | | | |
| methacrylate | | | | | |
| copolymer | | | | | |
| Further ingredients | ad 100 | ad 100 | ad 100 | ad 100 | ad 100 |
| | Formula | Formula | Formula | Formula | Formula |
| | 11 | 12 | 13 | 14 | 15 |
| Polyalcohol a1)¹⁾ | 0.5 to 30 | 1.0 to 25 | 1.5 to 20 | 2.0 to 15 | 3.0 to 10 |
| Polymer a2)²⁾ | 0.1 to 20 | 0.5 to 15 | 1.0 to 12 | 1.0 to 10 | 2.0 to 7.0 |
| Alcohol | 20 to 95 | 40 to 90 | 45 to 85 | 65 to 85 | 70 to 80 |
| Further ingredients | ad 100 | ad 100 | ad 100 | ad 100 | ad 100 |
| | Formula | Formula | Formula | Formula | Formula |
| | 16 | 17 | 18 | 19 | 20 |
| Glycerol | 0.5 to 30 | 1.0 to 25 | 1.5 to 20 | 2.0 to 15 | 3.0 to 10 |
| octylacrylamide/ | 0.1 to 20 | 0.5 to 15 | 1.0 to 12 | 1.0 to 10 | 2.0 to 7.0 |
| acrylates/ | | | | | |
| butylaminoethyl | | | | | |
| methacrylate | | | | | |
| copolymer | | | | | |
| Ethanol | 20 to 95 | 40 to 90 | 45 to 85 | 65 to 85 | 70 to 80 |
| Further ingredients | ad 100 | ad 100 | ad 100 | ad 100 | ad 100 |
| | Formula | Formula | Formula | Formula | Formula |
| | 21 | 22 | 23 | 24 | 25 |
| Polyalcohol a1)¹⁾ | 0.5 to 30 | 1.0 to 25 | 1.5 to 20 | 2.0 to 15 | 3.0 to 10 |
| Polymer a2)²⁾ | 0.1 to 20 | 0.5 to 15 | 1.0 to 12 | 1.0 to 10 | 2.0 to 7.0 |
| Alcohol | 20 to 95 | 40 to 90 | 45 to 85 | 65 to 85 | 70 to 80 |
| Hydroxylamine | 0.05 to 10 | 0.1 to 8 | 0.2 to 5 | 0.3 to 3 | 0.4 to 2 |
| Further ingredients | ad 100 | ad 100 | ad 100 | ad 100 | ad 100 |
| | Formula | Formula | Formula | Formula | Formula |
| | 26 | 27 | 28 | 29 | 30 |
| Glycerol | 0.5 to 30 | 1.0 to 25 | 1.5 to 20 | 2.0 to 15 | 3.0 to 10 |
| octylacrylamide/ | 0.1 to 20 | 0.5 to 15 | 1.0 to 12 | 1.0 to 10 | 2.0 to 7.0 |
| acrylates/ | | | | | |
| butylaminoethyl | | | | | |
| methacrylate | | | | | |
| copolymer | | | | | |
| Ethanol | 20 to 95 | 40 to 90 | 45 to 85 | 65 to 85 | 70 to 80 |
| Aminomethyl propanol | 0.05 to 10 | 0.1 to 8 | 0.2 to 5 | 0.3 to 3 | 0.4 to 2 |
| Further ingredients | ad 100 | ad 100 | ad 100 | ad 100 | ad 100 |
| | Formula | Formula | Formula | Formula | Formula |
| | 31 | 32 | 33 | 34 | 35 |
| Polyalcohol a1)¹⁾ | 0.5 to 30 | 1.0 to 25 | 1.5 to 20 | 2.0 to 15 | 3.0 to 10 |
| Polymer a2)²⁾ | 0.1 to 20 | 0.5 to 15 | 1.0 to 12 | 1.0 to 10 | 2.0 to 7.0 |
| Alcohol | 20 to 95 | 40 to 90 | 45 to 85 | 65 to 85 | 70 to 80 |
| Hydroxylamine | 0.05 to 10 | 0.1 to 8 | 0.2 to 5 | 0.3 to 3 | 0.4 to 2 |
| Surfactant/emulsifier | 0.01 to 5.0 | 0.02 to 4.0 | 0.03 to 3.0 | 0.05 to 2.0 | 0.1 to 1.0 |
| Further ingredients | ad 100 | ad 100 | ad 100 | ad 100 | ad 100 |
| | Formula | Formula | Formula | Formula | Formula |
| | 36 | 37 | 38 | 39 | 40 |
| Glycerol | 0.5 to 30 | 1.0 to 25 | 1.5 to 20 | 2.0 to 15 | 3.0 to 10 |
| octylacrylamide/ | 0.1 to 20 | 0.5 to 15 | 1.0 to 12 | 1.0 to 10 | 2.0 to 7.0 |
| acrylates/ | | | | | |
| butylaminoethyl | | | | | |
| methacrylate | | | | | |
| copolymer | | | | | |
| Ethanol | 20 to 95 | 40 to 90 | 45 to 85 | 65 to 85 | 70 to 80 |
| Aminomethyl propanol | 0.05 to 10 | 0.1 to 8 | 0.2 to 5 | 0.3 to 3 | 0.4 to 2 |
| PEG/PPG-n/m | 0.01 to 5.0 | 0.02 to 4.0 | 0.03 to 3.0 | 0.05 to 2.0 | 0.1 to 1.0 |
| dimethicone with n + m | | | | | |
| between 26 and 46 | | | | | |
| Further ingredients | ad 100 | ad 100 | ad 100 | ad 100 | ad 100 |
| | Formula | Formula | Formula | Formula | Formula |
| | 41 | 42 | 43 | 44 | 45 |
| Polyalcohol a1)¹⁾ | 0.5 to 30 | 1.0 to 25 | 1.5 to 20 | 2.0 to 15 | 3.0 to 10 |
| Polymer a2)²⁾ | 0.1 to 20 | 0.5 to 15 | 1.0 to 12 | 1.0 to 10 | 2.0 to 7.0 |
| Alcohol | 20 to 95 | 40 to 90 | 45 to 85 | 65 to 85 | 70 to 80 |
| Hydroxylamine | 0.05 to 10 | 0.1 to 8 | 0.2 to 5 | 0.3 to 3 | 0.4 to 2 |
| PEG/PPG-n/m | 0.01 to 5.0 | 0.02 to 4.0 | 0.03 to 3.0 | 0.05 to 2.0 | 0.1 to 1.0 |
| dimethicone with n + m | | | | | |
| between 26 and 46 | | | | | |
| Water | 0.5 to 20 | 1.0 to 15 | 1.0 to 10 | 1.5 to 8 | 2.0 to 6 |
| Further ingredients | ad 100 | ad 100 | ad 100 | ad 100 | ad 100 |
| | Formula | Formula | Formula | Formula | Formula |
| | 46 | 47 | 48 | 49 | 50 |
| Polyalcohol a1)¹⁾ | 0.5 to 30 | 1.0 to 25 | 1.5 to 20 | 2.0 to 15 | 3.0 to 10 |
| Polymer a2)²⁾ | 0.1 to 20 | 0.5 to 15 | 1.0 to 12 | 1.0 to 10 | 2.0 to 7.0 |
| Alcohol | 20 to 95 | 40 to 90 | 45 to 85 | 65 to 85 | 70 to 80 |
| Hydroxylamine | 0.05 to 10 | 0.1 to 8 | 0.2 to 5 | 0.3 to 3 | 0.4 to 2 |
| PEG/PPG-n/m | 0.01 to 5.0 | 0.02 to 4.0 | 0.03 to 3.0 | 0.05 to 2.0 | 0.1 to 1.0 |
| dimethicone with n + m | | | | | |
| between 26 and 46 | | | | | |
| PEG-60 hydrogenated | 0.01 to 3 | 0.02 to 2 | 0.05 to 1.5 | 0.1 to 1 | 0.2 to 0.6 |
| castor oil | | | | | |
| Perfume | 0.01 to 2 | 0.02 to 1.5 | 0.05 to 1.2 | 0.1 to 1 | 0.2 to 0.5 |
| Water | 0.5 to 20 | 1.0 to 15 | 1.0 to 10 | 1.5 to 8 | 2.0 to 6 |
| Further ingredients | ad 100 | ad 100 | ad 100 | ad 100 | ad 100 |
| | Formula | Formula | Formula | Formula | Formula |
| | 51 | 52 | 53 | 54 | 55 |
| Glycerol | 0.5 to 30 | 1.0 to 25 | 1.5 to 20 | 2.0 to 15 | 3.0 to 10 |
| octylacrylamide/ | 0.1 to 20 | 0.5 to 15 | 1.0 to 12 | 1.0 to 10 | 2.0 to 7.0 |
| acrylates/ | | | | | |
| butylaminoethyl | | | | | |
| methacrylate | | | | | |
| copolymer | | | | | |
| Ethanol | 20 to 95 | 40 to 90 | 45 to 85 | 65 to 85 | 70 to 80 |
| Aminomethyl propanol | 0.05 to 10 | 0.1 to 8 | 0.2 to 5 | 0.3 to 3 | 0.4 to 2 |
| PEG/PPG-18/18 | 0.01 to 0.5 | 0.02 to 4.0 | 0.03 to 3.0 | 0.05 to 2.0 | 0.1 to 1.0 |
| dimethicone | | | | | |
| PEG-60 hydrogenated | 0.01 to 3 | 0.02 to 2 | 0.05 to 1.5 | 0.1 to 1 | 0.2 to 0.6 |
| castor oil | | | | | |
| Cyclomethicone | <3.0 | <2.0 | <1.0 | <1.0 | — |
| Water | 0.5 to 20 | 1.0 to 15 | 1.0 to 10 | 1.5 to 8 | — |
| Perfume | 0.01 to 2 | 0.02 to 1.5 | 0.05 to 1.2 | 0.1 to 1 | 0.2 to 0.5 |
| Further ingredients | ad 100 | ad 100 | ad 100 | ad 100 | ad 100 |

¹⁾Polyalcohol a1) is selected from the group comprising glycerol, propylene glycol, and panthenol, in particular glycerol,
²⁾Polymer a2) is a polymer including carboxylic acid groups, wherein at least 50 wt % of the polymer is a copolymer formed by the monomers a2i) N-tert.-octylacrylamide, a2ii) acrylic acid, a2iii) tert.-butylaminoethylmethacrylic acid and optionally further monomers.

The ratio of the parts by weight of component a1) and component a2) in the styling product is greater than 1.

The aerosol products can be produced in the usual way. All constituents of the particular (propellant-free) styling product are introduced into a suitable pressure-resistant aerosol container. This is then sealed by a valve. The desired amount of propellant is lastly filled via conventional techniques. Propellants according to the invention are selected from hydrocarbons having 3 to 5 carbon atoms, such as propane, n-butane, iso-butane, n-pentane and iso-pentane, dimethyl ether, carbon dioxide, nitrous oxide, fluorocarbons and chlorofluorocarbons and mixtures of these substances and are used in amounts from 10 to 85 wt %, preferably 20 to 80 wt %, and in particular 35 to 60 wt %, based on the total aerosol composition.

The aerosol compositions, uses and methods according to the invention and some of their preferred embodiments are characterised by the following points:

1. An aerosol composition comprising
a) a cosmetic styling product including
   a1) at least one polyalcohol selected from the group consisting of glycerol, propylene glycol and panthenol, in particular glycerol,
   a2) polymer including carboxylic acid groups, wherein at least 50 wt % of the polymer including carboxylic acid groups is a copolymer formed by the monomers
      a2i) N-tert.-octylacrylamide,
      a2ii) acrylic acid,
      a2iii) tert.-butylaminoethylmethacrylic acid
      and optionally further monomers,
   characterised in that the ratio of the parts by weight of component a1) and component a2) in the product is greater than 1,
b) a propellant or mixture of propellants.

2. The aerosol composition according to item 1, characterised in that at least 60 wt %, preferably at least 70 wt %, particularly preferably at least 90 wt %, and in particular at least 95 wt %, and very particularly 100 wt % of the polymer a2) is a copolymer formed by the monomers
a2i) N-tert.-octylacrylamide,
a2ii) acrylic acid,
a2iii) tert.-butylaminoethylmethacrylic acid
and optionally further monomers.

3. The aerosol composition according to either one of the preceding items, characterised in that the ratio of the parts by weight of component a1) and component a2) in the cosmetic styling product is between 1:1 and 20:1, preferably between 2:1 and 10:1.

4. The aerosol composition according to any one of the preceding items, characterised in that the cosmetic styling product includes glycerol as component a1).

5. The aerosol composition according to any one of the preceding items, characterised in that the part by weight of component a1) in the total weight of the cosmetic styling product is between 0.5 and 30 wt %, preferably between 1.0 and 25 wt %, and in particular between 2.0 and 15 wt %.

6. The aerosol composition according to any one of the preceding items, characterised in that it includes, as component a2), at least one polymer known under the INCI name: octylacrylamide/acrylates/butylaminoethyl methacrylate copolymer.

7. The aerosol composition according to any one of the preceding items, characterised in that the part by weight of component a2) in the total weight of the cosmetic styling product is between 0.1 and 20 wt %, preferably between 0.5 and 15.0 wt %, and in particular between 1.0 and 10.0 wt %.

8. The aerosol composition according to any one of the preceding items, characterised in that the cosmetic styling product includes glycerol as component a1) and octylacrylamide/acrylates/butylaminoethyl methacrylate copolymer as component a2), wherein the ratio of the parts by weight of component a1) and component a2) in the product is greater than 1.

9. The aerosol composition according to any one of the preceding items, characterised in that the propellant or mixture of propellants is selected from dimethyl ether, alkanes having 3 to 5 carbon atoms, and fluorocarbons.

10. The aerosol composition according to any one of the preceding items, characterised in that the propellant or mixture of propellants is used in amounts from 10 to 85 wt %, preferably 20 to 80 wt %, and in particular 35 to 60 wt %, based on the total aerosol composition.

11. The aerosol composition according to any one of the preceding items, characterised in that the aerosol composition is present packaged in an aerosol container having a spraying device.

12. The aerosol composition according to any one of the preceding items, characterised in that the cosmetic styling product, based on its total weight, includes 0.01 to 5.0 wt %, preferably 0.02 to 4.0 wt %, and in particular 0.05 to 2.0 wt % of surfactants and/or emulsifiers.

13. The aerosol composition according to any one of the preceding items, characterised in that the cosmetic styling product, based on its total weight, includes 20 to 95 wt %, preferably from 40 to 90 wt %, and in particular from 65 to 85 wt % of an organic solvent different from the propellant (mixture).

14. The aerosol composition according to any one of the preceding items, characterised in that the cosmetic styling product, based on its total weight, has a water content from 0.5 to 20 wt %, preferably from 1.0 to 15 wt %, and in particular from 1.0 to 10 wt %.

15. Use of the aerosol composition according to any one of items 1 to 14 for the temporary deformation of keratin fibres, wherein the sheen effect, the volume and the feel of the hair are improved.

16. A method for treating hair, in which an aerosol composition according to any one of items 1 to 14 is applied to the hair and is left there until the hair is next washed.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An aerosol composition comprising
a) a cosmetic styling product including
a1) at least one polyalcohol selected from the group consisting of glycerol, propylene glycol and panthenol,
a2) polymer including carboxylic acid groups, wherein at least 50 wt % of the polymer including carboxylic acid groups is a copolymer formed by the monomers
a2i) N-tert.-octylacrylamide,
a2ii) acrylic acid,
a2iii) tert.-butylaminoethylmethacrylic acid
and optionally further monomers,
characterised in that the ratio of the parts by weight of component a1) and component a2) in the product is greater than 1,
b) a propellant or mixture of propellants.

2. The aerosol composition according to claim 1, wherein the styling product comprises at least 60 wt % of a copolymer formed by the monomers
a2i) N-tert.-octylacrylamide,
a2ii) acrylic acid,
a2iii) tert.-butylaminoethylmethacrylic acid
and optionally further monomers.

3. The aerosol composition according to claim 1, wherein the styling product comprises at least 90 wt % of a copolymer formed by the monomers
a2i) N-tert.-octylacrylamide,
a2ii) acrylic acid,
a2iii) tert.-butylaminoethylmethacrylic acid
and optionally further monomers.

4. The aerosol composition according to claim 1, wherein the styling product comprises at least 60 wt % of a copolymer formed by the monomers
a2i) N-tert.-octylacrylamide,
a2ii) acrylic acid,
a2iii) tert.-butylaminoethylmethacrylic acid
and optionally further monomers.

5. The aerosol composition according to claim 1, wherein the weight ratio of component a1) and component a2) in the styling product is between 1:1 and 20:1.

6. The aerosol composition according to claim 1, wherein the weight ratio of component a1) and component a2) in the styling product is between 2:1 and 10:1.

7. The aerosol composition according to claim 1, wherein the cosmetic styling product includes glycerol in component a1).

8. The aerosol composition according to claim 1, wherein component a1) comprises 0.5 to 30 wt % of the cosmetic styling product.

9. The aerosol composition according to claim 1, wherein component a1) comprises 1.0 and 25 wt % of the cosmetic styling product.

10. The aerosol composition according to claim 1, wherein component a1) comprises 2.0 and 15 wt % of the cosmetic styling product.

11. The aerosol composition according claim 1, wherein component a2) includes at least one polymer known under the INCI name: octylacrylamide/acrylates/butylaminoethyl methacrylate copolymer.

12. The aerosol composition according to claim 1, wherein component a2) comprises 0.1 and 20 wt % of the cosmetic styling product.

13. The aerosol composition according to claim 1, wherein component a2) comprises 0.5 and 15.0 wt % of the cosmetic styling product.

14. The aerosol composition according to claim 1, wherein component a2) comprises 1.0 and 10.0 wt % of the cosmetic styling product

15. The aerosol composition according to claim 1, wherein the propellant or mixture of propellants comprises 10 to 85 wt % of the aerosol composition.

16. The aerosol composition according to claim 1, wherein the propellant or mixture of propellants comprises 20 to 80 wt % of the aerosol composition.

17. The aerosol composition according to claim 1, wherein the propellant or mixture of propellants comprises 35 to 60 wt % of the aerosol composition.

18. A method for treating hair, in which an aerosol composition according to claim 1 is applied to the hair and is left there until the hair is next washed.
